(19) United States
(12) Patent Application Publication
(10) Pub. No.: US 2024/0279193 A1
(43) Pub. Date: Aug. 22, 2024

(54) PROCESS FOR REDUCING THE AGING-RELATED DEACTIVATION OF HIGH SELECTIVITY ETHYLENE OXIDE CATALYSTS

(71) Applicant: Dow Global Technologies LLC, Midland, MI (US)

(72) Inventors: Wen -Sheng Lee, Midland, MI (US); Mark H. McAdon, Midland, MI (US); Edward M. Calverley, Midland, MI (US); Vera Santos Castro, Terneuzen (NL)

(21) Appl. No.: 18/571,443

(22) PCT Filed: Sep. 19, 2022
(86) PCT No.: PCT/US22/43931

§ 371 Date: Dec. 18, 2023

Related U.S. Application Data
(60) Provisional application No. 63/247,483, filed on Sep. 23, 2021.

(51) Int. Cl. C07D 301/10; B01J 23/68; B01J 37/24

(52) U.S. Cl. CPC C07D 301/10; B01J 23/688; B01J 37/24

(57) ABSTRACT

Disclosed herein are methods of improving the life of high selectivity, silver catalysts for making ethylene oxide. Ethylene and oxygen are reacted over the high efficiency catalyst with at least one organic chloride modifier, and during a catalyst aging period of no less than 0.03 kt ethylene oxide/cubic meter catalyst, the overall catalyst chloriding effectiveness never exceeds an efficiency-maximizing optimum overall catalyst chloriding effectiveness value that corresponds to a reference feed gas composition and a set of reference reaction condition values. Reaction temperature and/or feed gas oxygen concentration are adjusted to obtain or maintain a desired value of an ethylene oxide production parameter. Once the reaction temperature and/or oxygen concentration vary by a specified amount from their respective reference values in the set of reference reaction condition values, the overall catalyst chloriding effectiveness is changed to account for a shift in the optimum (efficiency-maximizing) value.

### TECHNICAL FIELD

This disclosure relates generally to processes for making ethylene oxide, and more specifically, to a method of operating ethylene oxide production processes that reduces aging-related deactivation of high selectivity ethylene oxide catalysts.

### BACKGROUND

This disclosure relates to a process for manufacturing ethylene oxide (EO). Ethylene oxide is used to produce ethylene glycol, which is used as an automotive coolant, as antifreeze, and in preparing polyester fibers and resins, nonionic surfactants, glycol ethers, ethanolamines, and polyethylene polyether polyols.

The production of ethylene oxide generally occurs via the catalytic epoxidation of ethylene in the presence of oxygen. Conventional silver-based catalysts used in such processes provide a relatively low efficiency or “selectivity” (i.e., a lower percentage of the reacted ethylene is converted to the desired ethylene oxide). In certain exemplary processes, when using conventional catalysts in the epoxidation of ethylene, the theoretically maximal selectivity towards ethylene oxide, expressed as a fraction of the ethylene converted, does not reach values above the 6/7 or 85.7 percent limit. Therefore, this limit had long been considered to be the theoretically maximal selectivity of this reaction, based on the stoichiometry of the following reaction equation:

7C2H4+6 02→6C2H40+2CO2+2H2O

cf. the Kirk-Othmer Encyclopedia of Chemical Technology, 4th ed., Vol. No. 9, 1994, p. 926.

Certain “high efficiency” or “high selectivity” silver-based catalysts are highly selective towards ethylene oxide production. For example, when using certain catalysts in the epoxidation of ethylene, the theoretically maximal selectivity towards ethylene oxide can reach values above the 6/7 or 85.7 percent limit referred to, for example 88 percent, or 89 percent, or above. High selectivity catalysts comprise as their active components silver, rhenium, and at least one further metal. See EP0352850B1 and WO2007/123932.

Conventional catalysts have relatively flat selectivity curves with respect to the gas phase promoter concentration in the feed, i.e., the selectivity is almost invariant (i.e., the change in selectivity with respect to a change in gas phase promoter concentration in the feed is less than about 0.1%/ppmv) over a wide range of such promoter concentrations, and this invariance is substantially unaltered as reaction temperature is changed during prolonged operation of the catalyst. However, conventional catalysts have nearly linear activity decline curves with respect to the gas phase promoter concentration in the feed, i.e., with increasing gas phase promoter concentration in the feed, temperature has to be increased or the ethylene oxide production rate will be reduced. Therefore, when using a conventional catalyst, for optimum selectivity, the gas phase promoter concentration in the feed can be chosen at a level at which the maximum selectivity can be maintained at relatively low operating temperatures. Typically, the gas phase promoter concentration can remain substantially constant during the entire lifetime of a conventional catalyst. For conventional catalysts, the reaction temperature may be adjusted to obtain a desired production rate without any substantial need to adjust the gas phase promoter concentration.

By contrast, high selectivity catalysts tend to exhibit relatively steep selectivity curves as a function of gas phase promoter concentration as the concentration moves away from the value that provides the highest selectivity (i.e., the change in selectivity with respect to a change in gas phase promoter concentration is at least about 0.2%/ppmv when operating away from the selectivity maximizing promoter concentration). Thus, small changes in the promoter concentration can result in significant selectivity changes, and the selectivity exhibits a pronounced maximum, i.e., an optimum, at certain concentrations (or feed rates) of the gas phase promoter, when reactor pressure and feed gas composition are kept unchanged for a given reaction temperature and catalyst age.

For a high selectivity catalyst, at any given ethylene oxide production rate and set of operating conditions, a temperature (T) and overall catalyst chloriding effectiveness (Z*) combination exists that results in the maximum actual selectivity (“fixed production optimum”). This optimum is different than the efficiency-maximizing overall catalyst chloriding effectiveness value at a given temperature (“fixed temperature optimum”). However, both the fixed production optimum and the fixed temperature optimum are optimized based on selectivity. The overall catalyst chloriding effectiveness value at the temperature obtained from fixed production optimization is greater than the efficiency-maximizing, overall catalyst chloriding effectiveness value at that same temperature.

As is known in the art, the age of a catalyst can affect its activity due to a number of mechanisms. See Bartholomew, C. H., “Mechanisms of Catalyst Deactivation,” Applied Catalysis, A: General (2001), 212(1-2), 17-60. Catalyst age may be expressed in a number of ways such as days on stream or the ratio of cumulative product output (e.g., in metric kilotons, “kt”) divided by packed reactor volume (e.g., in cubic meters). All silver-based catalysts used in ethylene oxide production processes are subject to an aging-related performance decline during normal operation, and they need to be exchanged periodically. The aging manifests itself by a reduction in the activity of the catalyst and may also manifest itself by a reduction in selectivity. Usually, when a reduction in catalyst activity occurs, the reaction temperature is increased in order to maintain a constant ethylene oxide production rate. The reaction temperature may be increased until it reaches the design limit or becomes undesirably high, or the selectivity may become undesirably low, at which point in time the catalyst is deemed to be at the end of its lifetime and would need to be exchanged or regenerated. Current industry practice is to discharge and replace the catalyst when it is at the end of its useful life.

For high selectivity EO catalysts, several factors cause catalyst deactivation. The first is excessive chloride deposition on the catalyst surface due to decomposition of organic chloride gas phase promoters, such as ethyl chloride and dichloroethane, which in turn can result in the formation of silver chloride on the catalyst. The second is loss of silver surface area (decrease in silver dispersion) associated with silver particle coarsening (sintering). Other factors include vaporization or volatilization of silver, the formation of inactive phases, plugging with carbon deposits, and crushing, grinding, or erosion of the catalyst. For high selectivity EO catalysts, there is no apparent consensus as to the major factors that affect silver sintering. Nor is there any consensus on the impact of gas phase chloride promoter levels on activity aging. At least three patent publications—EP0352850(B1), WO2010123856, and WO2013058225—teach operating high-selectivity, silver EO catalysts at gas phase organic chloride moderator levels that exceed the levels that give peak efficiency. Thus, a need has arisen for a method of reducing the aging-related deactivation of a high-selectivity, rhenium-promoted, silver, ethylene oxide catalyst.

### SUMMARY

In accordance with the present disclosure, a method for reducing aging-related deactivation of a high-efficiency, rhenium-promoted silver catalyst in a process for manufacturing ethylene oxide is provided, wherein at the start of a first catalyst aging period the process has a first efficiency-maximizing, optimum overall catalyst chloriding effectiveness value at: a) a first reference feed gas composition, comprising ethylene at a first reference feed gas concentration value of ethylene, oxygen at a first reference feed gas concentration value of oxygen, water at a first reference feed gas concentration value of water, and at least one organic chloride at a first reference feed gas concentration value of the at least one organic chloride; and b) a first set of reference reaction condition values, comprising a first reference reaction temperature value, a first reference gas hourly space velocity value, and a first reference reaction pressure value. The method comprises reacting a first feed gas composition over the catalyst during the first catalyst aging period at: (i) a first overall catalyst chloriding effectiveness that never exceeds 95 percent of the first efficiency-maximizing, optimum overall catalyst chloriding effectiveness value during the first catalyst aging period; and (ii) a first set of reaction conditions, comprising a first reaction temperature that is no less than the first reference reaction temperature value and which varies from the first reference reaction temperature value by no more than +3° C. during the first catalyst aging period, the first reference reaction pressure value, and the first reference gas hourly space velocity value. The first feed gas composition comprises: aa) oxygen at a first feed gas concentration of oxygen that is no less than the first reference feed gas concentration value of oxygen, and which varies from the first reference feed gas concentration value of oxygen by no more than +1.2 volume percent during the first catalyst aging period, bb) ethylene at a first feed gas concentration of ethylene, and cc) water at a first feed gas concentration of water that is no greater than the first reference feed gas concentration value of water, and which varies from the first reference feed gas concentration value of water by no more than −0.4 volume percent during the first catalyst aging period, wherein the first catalyst aging period is no less than 0.03 kt ethylene oxide/m³ catalyst.

### BRIEF DESCRIPTION OF THE FIGURES

FIG. 1A is a process flow diagram depicting an embodiment of a process for making ethylene oxide by epoxidizing ethylene over a high selectivity silver-based catalyst comprising rhenium;

FIG. 1B is a plot of efficiency versus reactor outlet ethylene oxide concentration for three different reaction temperatures and four different overall catalyst chloriding effectiveness values used to illustrate fixed temperature optimization and fixed production optimization;

FIG. 2 is a flowchart depicting a method for reducing aging-related deactivation of a high-efficiency, rhenium-promoted, silver catalyst by reacting ethylene and oxygen over the catalyst at an underchlorided overall catalyst chloriding effectiveness value(s) to extend the useful life of the catalyst;

FIG. 3A is a plot of AEO versus time (t-to) used to illustrate a method of reducing aging-related deactivation for a high-selectivity, rhenium-promoted, silver ethylene oxide catalyst in accordance with Example 1;

FIG. 3B is a plot of reactor feed gas oxygen concentration versus time (t-to) used to illustrate a method of reducing aging-related deactivation of a high-selectivity, rhenium-promoted, silver ethylene oxide catalyst in accordance with Example 1;

FIG. 3C is a plot of reaction temperature versus time (t-to) used to illustrate a method of reducing aging-related deactivation of a high-selectivity, rhenium-promoted, silver ethylene oxide catalyst in accordance with Example 1;

FIG. 3D is a plot of Z* versus time (t-to) used to illustrate a method of reducing aging-related deactivation of a high-selectivity, rhenium-promoted, silver ethylene oxide catalyst in accordance with Example 1;

FIG. 3E is a plot of Z*/Z*opt vs. time (t-to) used to illustrate a method of reducing aging-related deactivation of a high-selectivity, rhenium-promoted, silver ethylene oxide catalyst in accordance with Example 1;

FIG. 3F is a plot of Asel vs. time (t-to) used to illustrate a method of reducing aging-related deactivation of a high-selectivity, rhenium-promoted, silver ethylene oxide catalyst in accordance with Example 1;

FIG. 3G is a plot of Asel vs. Z*/Z*opt used to illustrate a method of reducing aging-related deactivation of a high-selectivity, rhenium-promoted, silver ethylene oxide catalyst in accordance with Example 1.

FIGS. 4A-4F are plots of AEO versus time for six experimental runs from Example 2 in which ethylene oxide was produced at three different overall catalyst chloriding effectiveness values using six different microreactors;

FIGS. 5A-5F are plots of first-order GPLE models of AEO versus time for the six experimental runs shown in FIGS. 4A-4F.

FIGS. 6A-6F are plots of carbon efficiency versus time for the six runs of Example 2;

FIGS. 7A-7F are plots of root-mean-square (RMS) fitting errors of GPLE models as a function of the GPLE order parameter (0) for the six experimental runs shown in FIGS. 4A-4F;

FIG. 8A is a plot of average carbon efficiency versus the ratio (P) of the overall catalyst chloriding effectiveness value to the fixed temperature optimum overall catalyst chloriding effectiveness value for the six runs of Example 2;

FIG. 8B is a plot of gas hourly space velocity versus P for the six runs of Example 2;

FIG. 8C is a plot of the GPLE AEO(to) parameter vs. P for the six runs of Example 2;

FIG. 8D is a plot of the GPLE a parameter vs. P for the six runs of Example 2;

FIG. 8E is a plot of the GPLE L parameter vs. P for the six runs of Example 2;

FIGS. 9A-9C are plots of AEO (9A), work rate (9B), and relative catalyst activity=AEO(t)/AEO(t=2 days) (9C) versus time for five values of P=Z*/Z*opt, based on the first-order general power law equations;

### DETAILED DESCRIPTION

The present disclosure provides methods of operating a process for producing ethylene oxide by reacting ethylene, oxygen, and at least one organic chloride over a high-efficiency catalyst. The method comprises underchlorided operation of the process; i.e., at one or more sub-optimal overall catalyst chloriding effectiveness values relative to one or more fixed temperature, efficiency-maximizing optimum overall catalyst chloriding effectiveness values to reduce the aging-related deactivation of the catalyst and thereby extend its useful life. Without wishing to be bound by any theory, it is believed that operating the process in this underchlorided state extends the useful catalyst life due to a combination of avoiding excessive surface chloride and reducing the rate of silver sintering.

The present specification provides certain definitions to guide those of ordinary skill in the art in the practice of the present invention. Provision, or lack of provision, of a definition for a particular term or phrase is not meant to imply any particular importance, or lack thereof; rather, and unless otherwise noted, terms are to be understood according to the conventional usage by those of ordinary skill in the relevant art. Unless defined otherwise, technical and scientific terms used herein have the same meaning as commonly understood by one of skill in the art to which this invention belongs.

A supported catalyst for ethylene oxide manufacture should have acceptable activity, selectivity, and stability. One measure of the useful life of a catalyst is the length of time that reactants can be passed through the reaction system during which time acceptable productivity is obtained in light of all relevant factors.

The “activity” of a catalyst in a fixed bed reactor is generally defined as the reaction rate towards the desired product per unit of catalyst volume in the reactor. The activity of a catalyst can be quantified in a number of ways, one being the mole percent of ethylene oxide contained in the outlet stream of the reactor relative to that in the inlet stream (the mole percent of ethylene oxide in the inlet stream typically, but not necessarily, approaches zero percent) while the reaction temperature is maintained substantially constant; and another being the temperature required to maintain a given rate of ethylene oxide production. In many instances, activity is measured over a period of time in terms of the mole percent of ethylene oxide produced at a specified constant temperature. Alternatively, activity may be measured as a function of the temperature used to sustain production of a specified constant mole percent of ethylene oxide.

“AEO”, also referred to as “delta EO” or “AEO %”, is the difference between the outlet and inlet ethylene oxide concentrations, corrected for the change in molar volume across the reactor, measured in mole percent. It is calculated from the reactor inlet and outlet concentrations in mole percent of ethylene oxide (EOiniet and E0outiet, respectively) as follows: AEO %=SFE0outiet—Mulct. The term “SF” or “Shrink Factor” represents the net volumetric reduction occurring due to the production of the ethylene oxide. For every mole of ethylene oxide produced, there is a net reduction of 0.5 moles of total gas resulting in a corresponding reduction in the volumetric flow rate. The SF is typically calculated as follows: (200+EOiniet)/(200+EO outlet), where EOiniet and EOoutiet are the concentrations in mole percent of ethylene oxide in the reactor inlet and outlet gas mixtures, respectively.

Catalyst activity over life can be divided into two or three categories over time. Start-up occurs when there is a reactive mixture of oxygen and ethylene present. After the catalyst achieves an activity close to the production target, there can be a small, gradual increase in catalyst activity over a relatively short time interval relative to the useful catalyst life. Then the catalyst slowly starts to deactivate. For ethylene oxide catalysts under fixed operating conditions, catalyst activity aging may be represented by AEO(t)/AEO(t=tter) where tref s a reference time (e.g., days), or as AE0(x)/AE0(x=xter) where xref is a reference catalyst life in units of ethylene oxide production per unit volume of catalyst. Catalyst “activation” refers to the time period when catalyst activity is improving.

A catalyst “aging period” is a continuous period of time during which a catalyst is subjected to a reactive mixture of ethylene and oxygen. The aging period may be represented in units of time (e.g., days, weeks, years) or units of ethylene oxide mass production per unit volume of catalyst bed (e.g., kt ethylene oxide/m³ catalyst). At any time, the age of the catalyst is taken as the aggregate of all operations after 02 feeds are first initiated during startup of the fresh catalyst.

The “efficiency” of the oxidation, which is synonymous with “selectivity,” refers to the relative amount (as a fraction or in percent) of converted or reacted ethylene that forms a particular product. For example, the “selectivity to ethylene oxide” refers to the percentage on a molar basis of converted ethylene that forms ethylene oxide.

The term “ethylene oxide production parameter” is used herein to describe a variable that relates to the extent to which ethylene oxide is produced. Examples of ethylene oxide production parameters include ethylene oxide concentration, ethylene oxide yield, ethylene oxide production rate, ethylene oxide production rate/catalyst bed volume, ethylene conversion, and oxygen conversion. Thus, the ethylene oxide concentration relates to the ethylene oxide production rate because the production rate may be obtained by multiplying the ethylene oxide concentration and the net product flow rate from the reactor. The ethylene oxide production rate/catalyst bed volume may be determined by dividing the production rate by the volume of the catalyst bed. The oxygen and ethylene conversions are related to the production of the ethylene oxide by the selectivity. Selectivity and activity are not ethylene oxide production parameters. A “target ethylene oxide production parameter” is an ethylene oxide production parameter that is used as a specification for operating an ethylene oxide process. In one example, an ethylene oxide process is operated to achieve a specified value of an ethylene oxide production rate, in which case the ethylene oxide production rate would be considered a target ethylene oxide production parameter.

The term “first” when used in connection with reaction condition values, aging periods, feed gas concentration values, or optimum values is merely used to connote a time frame or aging period relative to a later time frame or aging period. “First” does not limit the scope of any particular claim to a fresh catalyst being started-up for the first time or to a start-up situation, generally. Similarly, the term “subsequent” is merely used to connote a time frame or aging period relative to an earlier time frame or aging period.

“Chloride-removing hydrocarbons” means hydrocarbons lacking chloride atoms.

These are believed to strip or remove chlorides from the catalyst. Examples include paraffinic compounds such as ethane and propane as well as olefins such as ethylene and propylene.

“Gas phase promoters” means compounds that enhance the selectivity and/or activity of a process for the production of ethylene oxide. Preferred gas phase promoters include organic chlorides. More preferably, the gas phase promoter is at least one selected from the group consisting of methyl chloride, ethyl chloride, ethylene dichloride, vinyl chloride, and mixtures thereof. Ethyl chloride and ethylene dichloride are most preferred as the gas phase promoter fed into the process.

The terms “high efficiency catalyst” and “high selectivity catalyst” refer to a catalyst that is capable of producing ethylene oxide from the ethylene and oxygen at a selectivity greater than 85.7 percent. The observed actual selectivity of a high selectivity catalyst may fall below 85.7 percent under certain conditions based on process variables, catalyst age, and the like. However, if the catalyst is capable of achieving at least an 85.7 percent selectivity, at any point during its life, for example, under any set of reaction conditions, or by extrapolating lower efficiencies observed at two different oxygen conversions obtained by varying gas hourly space velocity to the limiting case of zero oxygen conversion, it is considered to be a high selectivity catalyst.

“Overall catalyst chloriding effectiveness” means the net effect of the promoting and non-promoting gas phase species in chloriding the catalyst.

The term “operating conditions”, as used herein, refers to reaction parameters that include reaction temperature, reactor inlet pressure, reactor outlet pressure, gas hourly space velocity; average pressure along the catalyst bed, and any of the ethylene oxide production parameters (as defined above).

“Reaction temperature,” or “(T)” refers to any selected temperature(s) that are directly or indirectly indicative of the catalyst bed temperature. In certain embodiments, the reaction temperature may be a catalyst bed temperature at a specific location in the catalyst bed. In other embodiments, the reaction temperature may be a numerical average of several catalyst bed temperature measurements made along one or more catalyst bed dimensions (e.g., along the length). In additional embodiments, the reaction temperature may be the reactor outlet gas temperature. In further embodiments, the reaction temperature may be the reactor coolant outlet temperature. In other embodiments, the reaction temperature may be the reactor coolant inlet temperature.

The term “fixed production optimum” when used herein to describe an ethylene oxide process employing a high selectivity catalyst refers to a combination of values of reaction temperature and overall catalyst chloriding effectiveness that yields a maximum value for selectivity at a target value of a selected ethylene oxide production parameter while holding constant all of an ethylene concentration, an oxygen concentration, a carbon dioxide concentration, a reactor pressure, and a gas hourly space velocity, wherein each of the conditions may be measured as a reactor inlet, reactor outlet, or average catalyst bed value. In preferred examples, all of an ethylene concentration, an oxygen concentration, a water concentration, a carbon dioxide concentration, a reactor pressure, and a gas hourly space velocity are measured as reactor inlet values.

The term “fixed temperature optimum” when used herein to describe an ethylene oxide process employing a high selectivity catalyst refers to an overall catalyst chloriding effectiveness value that yields a maximum value for selectivity while holding constant all of reaction temperature, an ethylene concentration, an oxygen concentration, a water concentration, a carbon dioxide concentration, a reactor pressure, and a gas hourly space velocity, wherein each of the conditions may be measured as a reactor inlet, reactor outlet, or average catalyst bed value. In preferred examples, all of an ethylene concentration, an oxygen concentration, a water concentration, a carbon dioxide concentration, a reactor pressure, and a gas hourly space velocity are measured as reactor inlet values. Unless otherwise specified herein, the term “optimum” refers to a fixed temperature optimum.

The term “underchlorided” when used herein to describe an ethylene oxide process employing a high selectivity catalyst refers to refers to operation at an overall catalyst chloriding effectiveness value that is less than the fixed temperature optimum overall catalyst chloriding effectiveness value, i.e., a “sub-optimal” value of the overall catalyst chloriding effectiveness. In contrast, the term “overchlorided” when used herein to describe an ethylene oxide process employing a high selectivity catalyst refers to operation at an overall catalyst chloriding effectiveness value that is greater than the fixed temperature optimum overall catalyst chloriding effectiveness value, i.e., a “supra-optimal” value of the overall catalyst chloriding effectiveness.

The “work rate” of an ethylene oxide catalyst is the rate of change of the cumulative mass of ethylene oxide produced by the catalyst divided by the catalyst bed volume with respect to time and may be calculated as follows:

WR=[d(cumE0)/dt]/Vrx=GHSV·(MWEo/Vm)·(AEO/100 mol %) (1)
where, WR=work rate (kt EO/hr·m³);
   GHSV=gas hourly space velocity (hr⁻¹)=Tflow/Vrx;
   Tflow=inlet total flow rate in units of standard volume per hour;
   cumEO=cumulative mass of EO produced by catalyst (kt);
   Vrx=catalyst bed volume (m³)
   MWEo=molecular weight of EO=44.052.10⁻⁹ kt/gmol; and
   Vm=ideal gas volume at 0° C. and 1 atm (0.022414 m³/gmol)

High selectivity silver-based catalysts comprising rhenium and methods of making them are known to those of skill in the art. See EP0352850B1, WO2007/123932, WO2014/150669, EP1613428, or CN102133544.

Suitable reactors for the epoxidation reaction include fixed bed reactors, fixed bed tubular reactors, continuous stirred tank reactors (CSTR), fluid bed reactors and a wide variety of reactors that are well known to those skilled in the art. The desirability of recycling unreacted feed, or employing a single-pass system, or using successive reactions to increase ethylene conversion by employing reactors in series arrangement can also be readily determined by those skilled in the art. The epoxidation reaction is carried out at a temperature that is preferably at least about 200° C., more preferably at least about 210° C., and most preferably at least about 220° C.

Reaction temperatures of no more than about 300° C. are preferred, more preferably not more than about 290° C., and most preferably not more than about 280° C.

The reactor pressure is selected based on the desired mass velocity and productivity and ranges generally from about 5 atm (506 kPa) to about 30 atm (3.0 MPa). The gas hourly space velocity (GHSV) is preferably greater than about 3,000 hr⁻¹, more preferably greater than about 4,000 hr⁻¹, and most preferably greater than about 5,000 hr⁻¹.

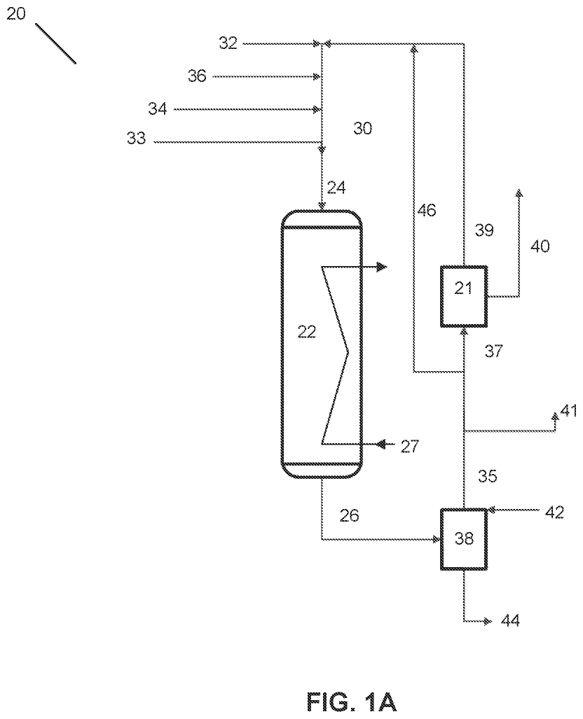

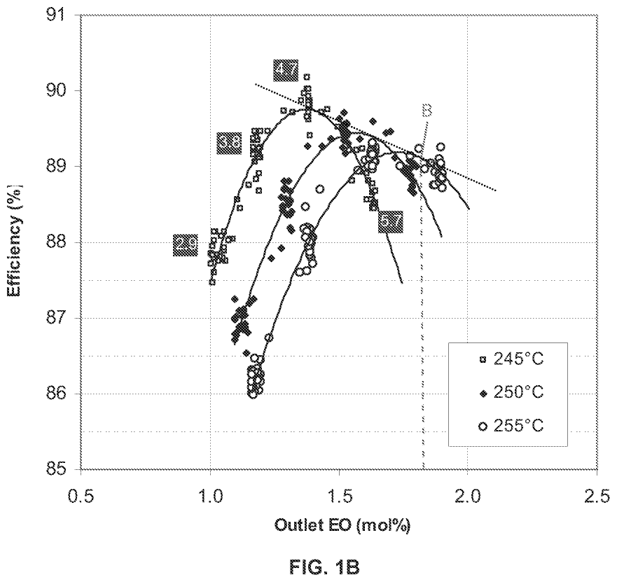

FIG. 1A is a process flow diagram depicting an embodiment of a process 20 for making ethylene oxide by epoxidizing ethylene over a high selectivity silver-based catalyst. Process 20 includes a reactor 22 comprising multiple reactor tubes with a high selectivity catalyst therein. Ethylene feed stream 36 (which may also include saturated hydrocarbons, such as ethane as an impurity), ballast gas 32, oxygen feed 34, and gas phase promoter make-up feed 33 each combine with recycle stream 30 to yield reactor feed gas inlet stream 24 proximate to the reactor 22 inlet. The reactor product stream 26 includes the ethylene oxide product in addition to side products (e.g., carbon dioxide, water, and small amounts of saturated hydrocarbons), unreacted ethylene, oxygen, and inert gases. The epoxidation reaction is generally exothermic. Thus, a coolant system 27 (e.g., a cooling jacket or a hydraulic circuit with a coolant fluid such as a heat transfer fluid or boiling water) is provided to regulate the temperature of reactor 22. The heat transfer fluid can be any of several well-known heat transfer fluids, such as tetralin (1,2,3,4Tetrahydronaphthalene).

The gas phase promoter in reactor feed 24 is generally a compound (or compounds) that enhances the efficiency and/or activity of process 20 (FIG. 1A) for producing the desired alkylene oxide. Preferred gas phase promoters include organic chlorides. More preferably, the gas phase promoter is at least one organic chloride selected from the group consisting of methyl chloride, ethyl chloride, ethylene dichloride, vinyl chloride, and mixtures thereof. Ethyl chloride and ethylene dichloride are most preferred as the make-up organic chloride in gas phase promoter feed 33. Using chlorohydrocarbon gas phase promoters as an example, it is believed that the ability of the promoter to enhance the performance (e.g., efficiency and/or activity) of process 20 for the desired alkylene oxide depends on the extent to which the gas phase promoter chlorinates the surface of the catalyst in reactor 22, for example, by depositing particular chlorine species such as atomic chlorine or chloride ions on the catalyst. However, hydrocarbons lacking chlorine atoms are believed to strip chlorides from the catalyst, and therefore, detract from the overall performance enhancement provided by the gas phase promoter. Discussions of this phenomenon can be found in Berty, “Inhibitor Action of Chlorinated Hydrocarbons in the Oxidation of Ethylene to Ethylene Oxide,” Chemical Engineering Communications, Vol. 82 (1989) at 229-232 and Berty, “Ethylene Oxide Synthesis,” Applied Industrial Catalysis, Vol. 1 (1983) at 207-238. Paraffinic compounds, such as ethane or propane, are believed to be especially effective at stripping chlorides from the catalyst. However, olefins such as ethylene and propylene, are also believed to act to strip chlorides from the catalyst. Some of these hydrocarbons may also be introduced as impurities in the ethylene feed 36 and/or ballast gas feed 32 or may be present for other reasons (such as the use of recycle stream 30). Typically, the preferred concentration of ethane in the reactor feed 24, when present, is from 0 to about 2 mole percent.

Given the competing effects of the gas phase promoter and the chloride-removing hydrocarbons in reactor feed stream 24, it is convenient to define an “overall catalyst chloriding effectiveness” that represents the net effect of gas phase species in chloriding the catalyst. In the case of organic chloride gas-phase promoters, the overall catalyst chloriding effectiveness can be defined as the dimensionless quantity Z* and represented by the following formula:

Z*=ethyl chloride equivalent (ppmv) ethane equivalent (mole percent) (2)

wherein the ethyl chloride equivalent is the concentration in ppmv (which is equivalent to ppm mole) of ethyl chloride that provides substantially the same catalyst chloriding effectiveness of the organic chlorides present in reactor feed stream 24 at the concentrations of the organic chlorides in feed stream 24; and the ethane equivalent is the concentration of ethane in mole percent that provides substantially the same catalyst dechloriding effectiveness of the non-chloride containing hydrocarbons in the reactor feed stream 24 at the concentrations of the non-chloride containing hydrocarbons in the reactor feed stream 24.

If ethyl chloride is the only gaseous chloride-containing promoter present in reactor feed stream 24, the ethyl chloride equivalent (i.e., the numerator in equation (2)) is the ethyl chloride concentration in ppmv. If other chlorine-containing promoters (specifically vinyl chloride, methyl chloride or ethylene dichloride) are used alone or in conjunction with ethyl chloride, the ethyl chloride equivalent is the concentration of ethyl chloride in ppmv plus the concentrations of the other gaseous chloride-containing promoters (corrected for their effectiveness as a promoter as compared to ethyl chloride). The relative effectiveness of a non-ethyl chloride promoter can be measured experimentally by replacing ethyl chloride with the other promoter and determining the concentration needed to obtain the same level of catalyst performance provided by ethyl chloride. As a way of further illustration, if the required concentration of ethylene dichloride at the reactor inlet is 0.5 ppmv to realize equivalent effectiveness in terms of catalyst performance provided by 1 ppmv ethyl chloride, then the ethyl chloride equivalent for 1 ppmv ethylene dichloride would be 2 ppmv ethyl chloride. For a hypothetical feed of 1 ppmv ethylene dichloride and 1 ppmv ethyl chloride, the ethyl chloride equivalent in the numerator of Z* would then be 3 ppmv. As a further example, it has been found that for certain catalysts methyl chloride has about 10 times less the chloriding effectiveness of ethyl chloride. Therefore, for such catalysts the ethyl chloride equivalent for a given concentration of methyl chloride in ppmv is 0.1×(methyl chloride concentration in ppmv). It has also been found that for certain catalysts, vinyl chloride has the same chloriding effectiveness as ethyl chloride. Therefore, for such catalysts the ethyl chloride equivalent for a given concentration of vinyl chloride in ppmv is 1.0×(vinyl chloride concentration in ppmv). When more than two chlorine-containing promoters are present in reactor feed stream 24, which is often the case in commercial ethylene epoxidation processes, the overall ethyl chloride equivalent is the sum of the corresponding ethyl chloride equivalents for each individual chlorine-containing promoter that is present. As an example, for a hypothetical feed of 1 ppmv ethylene dichloride, 1 ppmv ethyl chloride, and 1 ppmv vinyl chloride, the ethyl chloride equivalent in the numerator of Z* would be 2*1+1+1*1=4 ppmv.

The ethane equivalent (i.e., the denominator in equation (2)) is the concentration of ethane in mole percent in reactor feed stream 24 plus the concentration of the other hydrocarbons effective in removing chloride from the catalysts, corrected for their effectiveness for dechlorination relative to ethane. The relative effectiveness of ethylene compared to ethane can be measured experimentally by determining the inlet ethyl chloride equivalent concentration that provides the same level of catalyst performance for a feed comprising both ethylene and ethane as compared to the same feed with the same ethylene concentration but a specific ethyl chloride equivalent concentration and no ethane. As a way of further illustration, if with a feed composition comprising an ethylene concentration of 30.0 mole percent and an ethane concentration of 0.30 mole percent, a level of 6.0 ppmv ethyl chloride equivalents is found to provide the same level of catalyst performance as 3.0 ppmv ethyl chloride equivalents with a similar feed composition but lacking ethane, then the ethane equivalent for 30.0 mole percent ethylene would be 0.30 mole percent. For an inlet reactor feed 24 having 30.0 mole percent ethylene and 0.3 mole percent ethane, the ethane equivalent will then be 0.6 mole percent. As another illustration, it has been found that for certain catalysts methane has about 500 times less the dechloriding effectiveness of ethane. Thus, for such catalysts the ethane equivalent for methane is 0.002×(methane concentration in mol %). For a hypothetical inlet reactor feed 24 having 30.0 mole percent ethylene and 0.1 mole percent ethane, the ethane equivalent then will be 0.4 mole percent. For an inlet reactor feed 24 having 30.0 mole percent ethylene, 50 mole percent methane, and 0.1 mole percent ethane, the ethane equivalent then will be 0.5 mole percent. The relative effectiveness of hydrocarbons other than ethane and ethylene can be measured experimentally by determining the inlet ethyl chloride equivalent concentrations required to achieve the same catalyst performance for a feed comprising the hydrocarbon of interest at its concentration in the feed at two different concentrations of ethane in the feed. If a hydrocarbon compound is found to have a very small dechloriding effect and is also present in low concentrations, then its contribution to the ethane equivalent concentration in the Z* calculation may be negligible.

Thus, given the foregoing relationships, in the case where reactor feed stream 24 includes ethylene, ethyl chloride, ethylene dichloride, vinyl chloride, and ethane, the overall catalyst chloriding effectiveness value of process 20 can be defined as follows:

Z*=(ECL+2·EDC+VCL)+(C2H6+0.01·C2H4) (3)
wherein ECL, EDC, and VCL are the concentrations in ppmv of ethyl chloride (C2H5C1), ethylene dichloride (Cl—CH2-CH2-Cl), and vinyl chloride (H2C═CH—Cl), respectively, in reactor feed stream 24. C2H6 and C2H4 are the concentrations in mole percent of ethane and ethylene, respectively, in reactor feed stream 24. It is important that the relative effectiveness of the gaseous chlorine-containing promoter and the hydrocarbon dechlorinating species also be measured under the reaction conditions which are being used in the process. Z* will preferably be maintained at a level that is no greater than about 20 and which is most preferably no greater than about 15. Z* is preferably at least about 1.

In preferred examples, only a single species of make-up organic chloride is supplied in gas phase promoter make-up feed 33. Although the gaseous chlorine-containing promoter may be supplied as a single species, upon contact with the catalyst, other species may be formed leading to a mixture in the gas phase. Consequently, if the reaction gases are recycled such as via recycle stream 30, a mixture of species will be found in the inlet of the reactor. In particular, the recycled reaction gases at the inlet may contain ethyl chloride, vinyl chloride, ethylene dichloride and methyl chloride, even though only ethyl chloride or ethylene dichloride is supplied to the system. The concentrations of ethyl chloride, vinyl chloride, and ethylene dichloride must be considered in calculating Z*.

Recycle stream 30 is provided to minimize waste and increase savings as the recycling of unreacted reactants decreases the amount of fresh “make up” feed (e.g., fresh alkylene, oxygen, and ballast gas) supplied to reactor 22. One example of a suitable recycle system is depicted in FIG. 1A. As shown in the figure, ethylene oxide absorber 38 includes a feed stream defined by reactor product stream 26 and also includes lean water feed stream 42. Ethylene oxide absorber 38 produces a rich water stream 44 and an overhead gas stream 35 that is an intermediate stream between ethylene oxide absorber 38 and carbon dioxide removal unit 21 and which comprises unreacted olefin, saturated hydrocarbon impurities or byproducts, and carbon dioxide. Carbon dioxide is removed in CO2 removal unit 21 (e.g., a CO2 scrubber coupled with a regenerator) and exits CO2 removal unit 21 in carbon dioxide stream 40. The overhead stream 39 from CO2 removal unit 21 is combined with CO2 removal unit 21 bypass stream 46 to define recycle stream 30. Purge line 41 is also provided to provide for the removal of saturated hydrocarbon impurities (e.g., ethane), inerts (such as argon), and/or byproducts (as well as carbon dioxide) to prevent their accumulation in reactor feed 24. CO2 removal unit 21 feed stream 37 is defined by ethylene oxide absorber 38 overhead stream 35, after accounting for CO2 removal unit 21 bypass stream 46, if present, and purge line 41.

Oxygen feed 34 may comprise substantially pure oxygen or air. Generally, the oxygen concentration in reactor feed 24 will be at least about 1 mole percent and preferably at least about 2 mole and percent. The oxygen concentration will generally be no more than about 15 mole and volume percent and preferably no more than about twelve (12) mole and volume percent. The ballast gas 32 (e.g., nitrogen or methane) is generally from about 50 mole and volume percent to about 80 mole and volume percent of the total composition of reactor feed stream 24.

The concentration of ethylene in reactor feed stream 24 may be at least about 18 mole percent and more preferably at least about 20 mole percent. The concentration of ethylene in reactor feed stream 24 is preferably no greater than about 50 mole percent, and more preferably is no greater than about 40 mole percent.

When present, the carbon dioxide concentration in reactor feed stream 24 has an adverse effect on the selectivity, activity and/or stability of catalysts used in reactor 22. Carbon dioxide is produced as a reaction by-product and may also be introduced with other inlet reaction gases as an impurity. In commercial ethylene epoxidation processes, at least a part of the carbon dioxide is removed continuously in order to control its concentration to an acceptable level in the cycle. The carbon dioxide concentration in reactor feed 24 is generally no more than about 8 mole percent, preferably no more than about 4 mole percent, and even more preferably no more than about 2 mole percent of the total composition of reactor feed gas stream 24. Water may also be present in the reactor feed gas stream 24 in a concentration that is up to 2 mole percent.

In an embodiment, the preferred concentration of ethane in the reactor feed 24, when present, is up to about 2 mole percent and may reach concentrations lower than 0.1 mole percent or even 0.05 mole percent.

Referring to FIG. 1B, a plot of efficiency versus reactor outlet ethylene oxide concentration is shown for three different reaction temperatures (245° C., 250° C., and 255° C.) and four different overall catalyst chloriding effectiveness values (Z*=2.9, 3.8, 4.7, and 5.7). As FIG. 1B indicates, increasing the reaction temperature shifts the parabolic relationship between efficiency and reactor outlet ethylene oxide concentration down and to the right. Increasing the value of Z* at a constant reaction temperature traverses the parabola corresponding to the current reaction temperature from the left to the right. A tangent line can be drawn to the parabolas and is shown in FIG. 1B. The tangent line defines the fixed production optimum combination of temperature and Z* for a desired reactor outlet ethylene oxide concentration. For a fixed temperature, the peak of a parabola corresponding to that temperature defines the fixed temperature optimum Z* value. The parabola that is furthest to the left and highest up in FIG. 1B corresponds to 245° C. and has a fixed temperature optimum Z* value of 4.7, which achieves an efficiency of about 89.7 percent. The parabola that is furthest to the right and lowest in FIG. 1B corresponds to a reaction temperature of 255° C. and has a fixed temperature optimum Z* value of about 5.2. At an outlet ethylene oxide concentration of about 1.8 mole percent, the fixed production optimum is defined by point B, which corresponds to a reaction temperature of about 255° C. and a Z* value of about 5.5. However, at that same reaction temperature, the fixed temperature optimum value of Z* is about 5.2, corresponding to a slightly lower outlet ethylene oxide concentration of about 1.7 mole percent.

The present disclosure resulted from the unexpected finding that operating at a Z* value that is less than the fixed temperature optimum Z* value (referred to as Z*opt herein) extends the useful life of high selectivity, ethylene oxide catalysts. In certain examples, the Z* value that extends the useful life is no greater than 95 percent, preferably no greater than 90 percent, and still more preferably no greater than about 85 percent of the fixed temperature optimum Z* value. In certain examples, operation at the sub-optimum Z* value is maintained for a catalyst aging period of at least about 0.03 kt ethylene oxide/m³ catalyst, preferably at least about 0.06 kt ethylene oxide/m³ catalyst, more preferably at least about 0.09 kt ethylene oxide/m³ catalyst, and still more preferably at least about 0.12 kt ethylene oxide/m³ catalyst. Operating at sub-optimum Z* values is preferably maintained for multiple catalyst aging periods, which may be contiguous or noncontiguous, during the life of a particular batch of catalyst. In certain examples, it is preferable to operate at sub-optimum Z* values for a cumulative aging period of at least 1 kt/m³ ethylene oxide production, more preferable to operate at sub-optimum Z* values for a cumulative aging period of at least 2 kt/m³ ethylene oxide production, and even more preferable to operate at sub-optimum Z* values for a cumulative aging period of at least 3 kt/m³ ethylene oxide production.

The fixed temperature optimum used to define sub-optimum Z* values corresponds to a set of reference reaction conditions. In preferred examples, the fixed temperature optimum defines an efficiency-maximizing, optimum overall catalyst chloriding effectiveness value Z*opt that corresponds to a reference feed gas composition and a first set of reference reaction condition values. The reaction reference condition values comprise a reference reaction temperature value, a reference gas hourly space velocity value, and a reference reaction pressure value. The reference feed gas composition comprises ethylene at a reference feed gas concentration value of ethylene, oxygen at a reference feed gas concentration value of oxygen, water at a reference feed gas concentration value of water, and at least one organic chloride at a reference feed gas concentration value of the at least one organic chloride. In certain preferred examples, Z* is maintained at a sub-optimum value based on the optimum value Z*opt that corresponds to a set of reference conditions and a reference feed gas composition for a catalyst aging period of no less than 0.03 kt ethylene oxide/m³ catalyst, preferably no less than 0.06 kt ethylene oxide/m³ catalyst, more preferably no less than 0.09 kt ethylene oxide/m³ catalyst, and still more preferably no less than 0.12 kt ethylene oxide/m³ catalyst. During that catalyst aging period, the reaction temperature is no less than the reference reaction temperature value and varies from the reference reaction temperature value by no more than +3° C. (preferably +2° C. and more preferably +1° C.), the feed gas concentration of oxygen is no less than the reference feed gas concentration value of oxygen and varies from the reference feed gas concentration value of oxygen by no more than +1.2 volume percent (preferably +0.8 volume percent and more preferably +0.4 volume percent), the feed gas concentration of water is no greater than the reference feed gas concentration value of water and varies from the reference feed gas concentration value of water by no more than −0.4 volume percent (preferably −0.3 volume percent and more preferably −0.2 volume percent), the reaction pressure is held at the reference reaction pressure, and the gas hourly space velocity is held at the reference gas hourly space velocity value.

A “selectivity penalty” may be defined as the difference in selectivity between operation at the fixed temperature optimum value of the overall catalyst chloriding effectiveness and operation at the selected sub-optimum value of the overall catalyst chloriding effectiveness. The methods described herein preferably decrease catalyst activity aging while incurring a minimal, initial selectivity penalty. In preferred examples, the initial selectivity penalty is no more than about 0.5%, preferably no more than about 0.4% and more preferably no more than about 0.2%.

In certain preferred examples of the methods described herein, it is desirable to maintain or adjust a value of an ethylene oxide production parameter. In preferred examples, at least one of reaction temperature and feed gas oxygen concentration is adjusted to maintain or adjust the value of an ethylene oxide production parameter. However, once the reaction temperature or feed gas oxygen concentration vary by more than a selected amount from their respective reference values (e.g., +3° C. or +1.2 volume percent, respectively), adjustments are preferably made to the overall catalyst chloriding effectiveness value to account for the fact that the optimum overall catalyst chloriding effectiveness value has shifted. This may entail using the current set of feed gas compositions and reaction conditions as reference conditions to determine a new fixed temperature optimum value of the overall catalyst chloriding effectiveness or using known correlations or rules of thumb for making the adjustment.

In certain examples, Z*opt is determined based on correlations between Z*opt and a set of reaction conditions comprising reaction temperature (T), oxygen concentration (Co2), and water concentration (C1-12o). In preferred examples, the correlation is a linear non-proportional correlation such as the following:

$$Z * opt = 5.3 + 0.1 ( T - 240 ° C . ) + 0.25 ( Co 2 - 8 vol . \% ) - 0.7 ( Cmo - 0.2 vol . \% ) . \qquad ( 4 )$$

The same method can then be repeated for subsequent aging periods, with Z* being adjusted when the reaction temperature and/or feed gas oxygen concentration deviate from their subsequent reference values by a specified amount. For high selectivity silver EO catalysts, after changes in certain parameters such as Z*, it can take 24-96 hours for the catalysts to achieve steady-state performance in activity and selectivity.

It has been found that, for high selectivity silver EO catalysts that are operated at fixed reaction conditions and fixed feed gas compositions, as the catalyst loses activity, the activity aging follows a first-order general power law equation of the type:

y(t)=AEO(t)=AEO(to)·[(100%−L)(exp(a·(t−to))+L] (5)

where, a=rate parameter (days⁻¹)

t=time (days)

L=asymptotic limit of y (in percent; dimensionless and non-negative)

AEO(t)=AEO at time t (mole percent)
AEO(to)=AEO at time to, where to is a reference time.

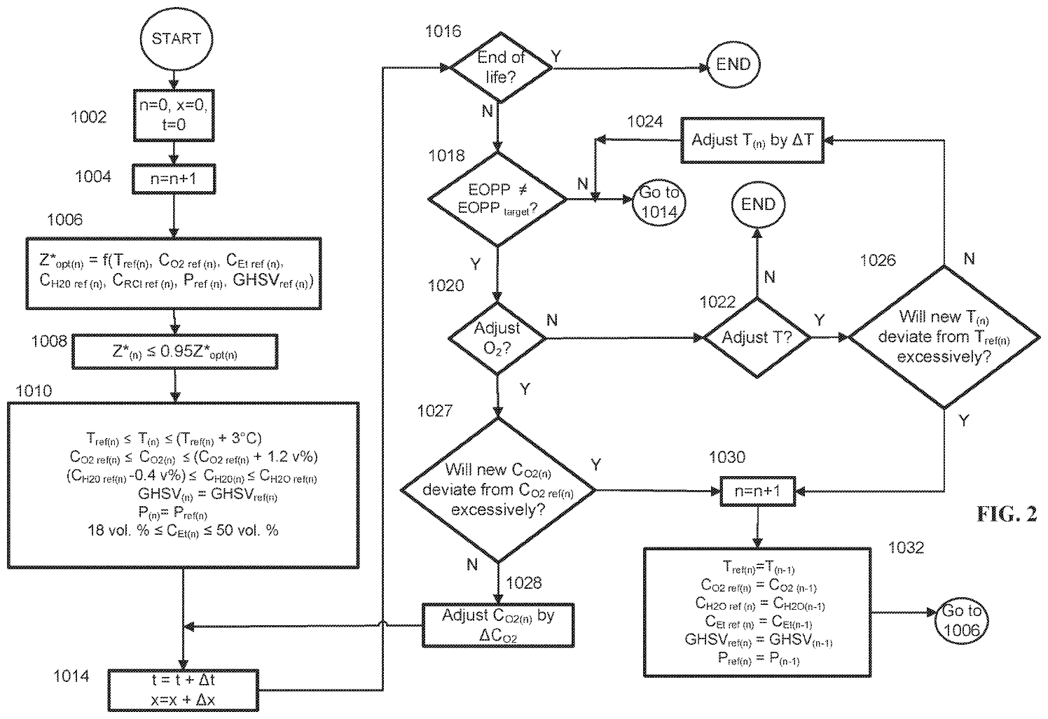

Referring to FIG. 2, a method for reducing aging-related deactivation of a high-efficiency, rhenium-promoted silver catalyst in a process for manufacturing ethylene oxide will now be described. In the method of FIG. 2, it is assumed that the reaction temperature would not be constrained at the same time the feed gas oxygen concentration reaches a flammability limit. However, it is understood that when feed gas oxygen concentration is adjusted to maintain a desired value of an ethylene oxide production parameter, a safe margin from the flammability limit will be maintained. The variable n is an aging period index used to distinguish periods in which there is a significant shift in the value of Z*opt, which may be known directly or via correlations. The aging period index n is initialized in step 1002 and incremented in step 1004. The elapsed aging counters x and t are also initialized in step 1002. The x counter is for aging periods expressed in units of mass of ethylene oxide per volume of catalyst bed, and the t counter is for aging periods expressed in units of time. The counters are incremented by respective selected increments Ax and At in step 1014. The increments are selected based on the frequency with which the various evaluation steps 1016, 1018, 1020, 1022, and 1026 can be carried out. Both counters are shown, but only one needs to be used.

In step 1006 there is an nth fixed-temperature optimum chloriding effectiveness parameter (Z*opt(n)) that corresponds to an nth set of reference reaction conditions and an nth reference feed gas composition. The nth set of reference reaction conditions are an nth reference reaction temperature (Tref(n)), an nth reference reaction pressure (1³ref(n), and an nth reference gas hourly space velocity (GHSVtor(o). The nth reference feed gas composition comprises ethylene at an nth reference feed gas concentration value of ethylene (CEt ref(n)), oxygen at an nth reference feed gas concentration value of oxygen (Co2ref(n)), water at an nth reference feed gas concentration value of water CH20 ref(n), and at least one organic chloride promoter R—Cl at an nth reference feed gas concentration of the at least one organic chloride promoter (CRC1 ref(n)). Step 1006 is not meant to imply that an optimization is necessarily carried out, but rather, that there is at this point in the process an nth fixed temperature optimum value of overall catalyst chloriding effectiveness and that it corresponds to an nth reference feed gas composition and an nth set of reference reaction conditions.

In step 1008 the nth overall catalyst chloriding effectiveness Z*(0 is set to a value that is no more than 0.95Z*opt(n), preferably no more than 0.90Z*opt(n), and more preferably no more than 0.85Z*optto. Z* may have additional values during each aging period (n), but they will not exceed 0.95·Z*optto, preferably not exceed 0.90·Z*opto, and more preferably not exceed

0.85.Z*opto. This step may be carried out by performing an optimization to determine Z*opt or by using a correlation of Z*opt and certain process variables.

In step 1010 the nth feed gas composition is then reacted over the high-efficiency catalyst at an nth set of reaction conditions during an nth catalyst aging period of at least 0.03 kt EO/m³ catalyst, preferably at least 0.06 kt EO/m³ catalyst, and more preferably at least 0.12 kt EO/m³ catalyst. The nth set of reaction conditions includes an nth reaction temperature, the nth reference reaction pressure value, and the nth reference gas hourly space velocity value. In step 1010, the parameters T(0, Co2(0, CH2o(n) are the current values of the reaction temperature, the feed gas concentration of oxygen, and the feed gas concentration of water.

The nth reaction temperature (T(0) may vary from the nth reference reaction temperature Tref(n) but will preferably be no less than Tref(n) and will not exceed Tref(n) by more than +3° C., preferably +1° C., more preferably +0.8° C., and still more preferably +0.4° C.

The nth feed gas composition comprises ethylene at a nth feed gas concentration of ethylene (Ca(n>) ranging between 18 and 50 volume percent of the total feed gas volume, oxygen at an nth feed gas concentration of oxygen (Co2(0), and water at an nth feed gas concentration of water (CH2o(o). Co2(0 may vary from the nth reference feed gas concentration of oxygen (CO2 ref(n)), but will preferably be no less than the nth reference feed gas concentration of oxygen (CO2ref(n)) and will not exceed Co2ref(n) by more than preferably +1.2 volume percent, more preferably +0.8 volume percent, and still more preferably +0.4 volume percent. CH2o(n) is preferably no greater than the nth reference feed gas concentration of water (CH20 ref(n)) and will not vary from CH20 ref(n) by more than preferably −0.4 volume percent, more preferably −0.3 volume percent, and still more preferably −0.2 volume percent.

In step 1016 it is determined whether the catalyst has reached its end of life in which case x and/or t have reached their maximum values. The “end of life” may be determined in a variety of different ways, including by using catalyst aging models alone or in conjunction with observed catalyst performance decline, equipment limitations, and the cost and availability of replacement catalyst. If the catalyst has reached end of life, the method ends. Otherwise, control transfers to step 1018.

In step 1018 a current value of an ethylene oxide production parameter (EOPP) is compared to its target value (EOPP target). If the current and target values do match (i.e., step 1018 returns a value of NO) or at least match within a specified tolerance, control transfers to step 1014 and the aging period counters Ax and At are incremented. Otherwise, control transfers to step 1020 and it is determined whether the feed gas oxygen concentration will be adjusted to achieve EOPP target. Step 1020 may itself comprise a number of other determination steps. In certain examples, if EOPP is less than EOPP target, a determination is made as to whether the current reactor feed gas oxygen concentration is at or will exceed the flammability limit after making the desired change in feed gas oxygen concentration (ACo2). If it does, then step 1020 returns a value of NO, and control transfers to step 1022.

If the current feed gas oxygen concentration value (Co2(0) is less than the flammability limit, then step 1020 returns a value of YES, and control transfers to step 1027. In step 1027, a determination is made whether incrementing the feed gas oxygen concentration by the desired change in feed gas oxygen concentration (ACo2) will not cause the resulting feed gas oxygen concentration (Co2(0+ACo2) to deviate “excessively” from the reference concentration (CO2ref(n)). In certain preferred examples, “excessively” in step 1027 means that the resulting feed gas oxygen concentration (Co2(0+ACo2) will exceed the reference feed gas oxygen concentration (CO2 ref(n)) by more than 1.2 volume percent or fall below the reference feed gas oxygen concentration (Co2 ref(n). If neither condition is true, step 1027 returns a value of NO, and control transfers to step 1028 to increment the feed gas oxygen concentration by ACo2 Otherwise step 1020 returns a value of YES, and control transfers to step 1030 to increment the aging index n and establish new reference reaction condition values and reference feed gas composition values in step 1032.

If step 1020 returns a value of NO, control transfers to step 1022. In step 1022, a determination is made as to whether the reaction temperature will be adjusted to achieve EOPPtarget. Step 1022 may itself include other determination steps. In the case of an EOPP value that is below EOPP target, a determination will be made as to whether a desired change in the reaction temperature (AT) will cause the resulting reaction temperature (T(o+AT) to exceed a maximum desirable or achievable reaction temperature value (e.g., based on equipment limitations, safety considerations, and/or catalyst performance considerations). If so, step 1022 returns a value of NO, and the method ends or EOPPtarget is reduced to an achievable value. If the resulting reaction temperature (T(o+AT) will not exceed the maximum desirable or achievable reaction temperature value, then step 1022 returns a value of YES, and control transfers to step 1026.

In step 1026 a determination is made as to whether incrementing the reaction temperature to achieve EOPPtarget will yield a resulting reaction temperature (T(o+AT) that will deviate excessively relative to some defined criteria. In one preferred example, “excessive” in step 1026 means either that the resulting reaction temperature (T(nrkAT) will fall below the reference reaction temperature (Tref(n)), or that it will exceed the reference reaction temperature value (Tref(n)) by more than 3° C. (preferably 2° C. and more preferably 1° C.). If either condition is true, step 1026 will return a value of YES, and control transfers to step 1030 to increment the aging index n and establish a new set of reference reaction conditions and a new reference feed gas composition. Step 1032. If the resulting reaction temperature (T(nrkAT) will not fall below the reference reaction temperature (Tref(n)), or exceed the reference reaction temperature value Tref(0) by more than 3° C. (preferably 2° C. and more preferably 1° C.), step 1026 returns a value of NO, and control transfers to step 1024 and the reaction temperature is increased by AT.

Returning to step 1020, if EOPP is greater than EOPPtarget, and if the current value of the reaction temperature (T(0) has not reached a minimum temperature constraint (e.g., based on a cooling circuit limit that makes any further temperature decreases unattainable or based on a catalyst limitation that makes any further decrease in reaction temperature undesirable), then step 1020 returns a value of NO and control transfers to step 1022. Otherwise, step 1020 returns a value of YES and control transfers to step 1027.

### Example 1 (Hypothetical)

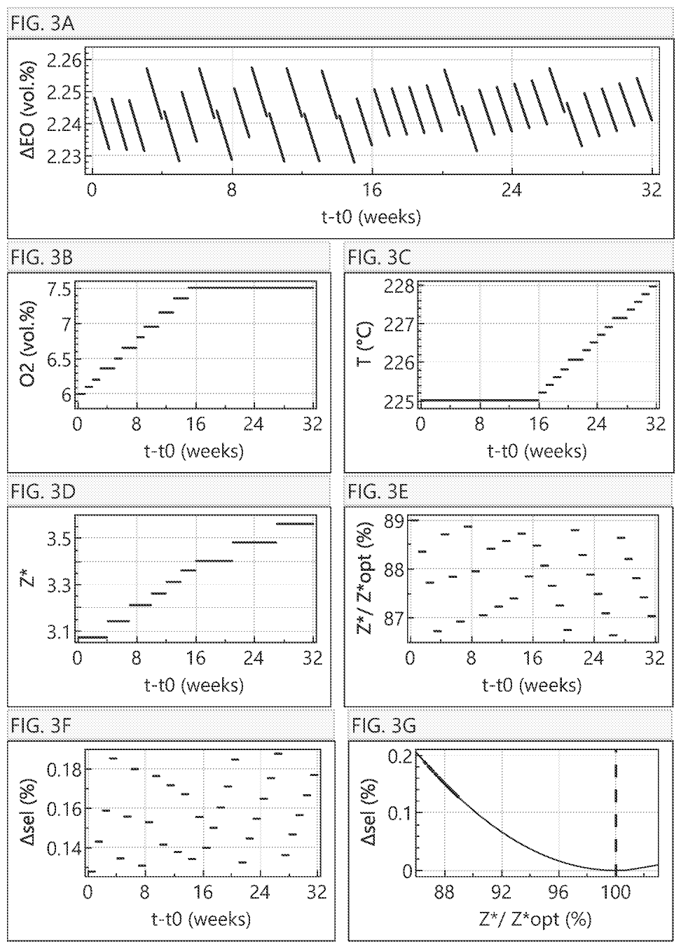

This hypothetical example illustrates a method of underchloriding a high-efficiency ethylene oxide catalyst to reduce aging-related catalyst deactivation, as shown in Table I and FIG. 3. In this example, the catalyst activity (AEO), the selectivity penalty (Asel), the EO work rate, and the cumulative EO production are computed as a function of time (t) using a set of equations. The following parameters are constant: GHSV=6000/hr., catalyst bed pressure=2.12 MPa, feed gas concentration of ethylene=30 vol. %, feed gas concentration of carbon dioxide=1.1 vol. %, and feed gas concentration of water (steam)=0.2 vol. %. The reaction temperature (T, in units of ° C.) and the feed gas concentration of oxygen (Co2, in units of vol. %) are varied over time in order to maintain a desired ethylene oxide production parameter (AEO); i.e., to compensate for deactivation of the catalyst.

In this example, the catalyst reaches steady performance and begins to age at a time t=to=11 days. At 11 days, the reaction temperature is T(to)=225° C., the feed gas concentration of oxygen is Co2(to)=6.0 vol. %, the chloriding effectiveness value is Z*(to)=3.07, and the ethylene oxide production parameter is AEO=2.25 vol. %. Starting at t=to=11 days, the hypothetical aging parameter AEO(t)/AEO(to) follows the well-known sintering, first order decay function of equation (5) multiplied by a rate function Q(t), where Q(t) is the product of an Arrhenius equation temperature dependence factor and feed gas concentration factors for oxygen and the chloriding effectiveness value, which is taken as Z*. The optimum value of Z* (i.e., Z*opt) depends on reaction temperature, feed gas oxygen concentration, and feed gas water concentration (fixed at CH2o=0.2 vol. %). The hypothetical selectivity penalty depends on the difference between Z* and Z*opt. The equations and parameters are as follows:

$$[ AEO ( t ) / AEO ( to ) ] 100 \% = [ ( 100 \% - L ) * exp ( - a . ( t - to ) ) + L ] Q ( t ) \qquad ( 6 )$$

$$Q ( t ) = exp [ - EA / ( R ( T ( t ) + 273.15 ) ) + EA / ( R ( T ( to ) + 273.15 ) ) ] ( CO 2 ( 0 / CO 2 ( t 0 ) ) ° ( Z * ( t ) / Z * ( to ) ) ze \qquad ( 7 )$$

$$Z * opt ( t ) = 5.3 + 0.1 . ( T ( t ) - 240 ° C . ) + 0.25 . ( Co 2 ( t ) - 8 vol . \% ) - 0.7 . ( CH 2 o ( t ) - 0.2 vol . \% ) \qquad ( 8 )$$

$$Asel = 1.05 . {( Z * / Z * opt - 1 )}^{2} , \qquad ( 9 )$$

where, in equation (6), the variable t and parameters {to, AE0(t0), a, L} have the same definitions as those of equation (5);
the aging parameters are taken as a=0.002/day and L=40%;

R=8.3145 J/K/mol is the ideal gas constant;
o=0.5 is the exponent that gives the dependence of aging rate on feed gas oxygen concentration;
ze=0.1 is the exponent that gives the dependence of aging rate on Z*; and EA=80 kJ/mole is the activation energy.

In order to compensate for the deactivation of the catalyst, each week one adjustment is made to either the feed oxygen concentration, or the reaction temperature or the chloriding effectiveness value (Z*), as shown in FIG. 3. Of these three target values, only one is adjusted each week. These adjustments are generally consistent with the method of FIG. 2. Every four weeks the data is reviewed, and adjustments are made prior to starting the next four-week aging period. These adjustments include the desired range of the ethylene oxide production parameter (AEO), the reference feed gas composition values, the reference reaction condition values, and the efficiency-maximizing, optimum overall catalyst chloriding effectiveness value.

In this example, the process is operated to maintain the ethylene oxide production parameter AEO(t) at desired values that are no less than 2.22 vol. % and no greater than 2.26 vol. % (FIG. 3A), and the feed gas oxygen concentration at values no greater than 7.5 vol. % (FIG. 3B).

The selectivity penalty is maintained with a range of 0.12-0.19 percent by adjusting Z* (FIGS. 3F, 3D, and 3G). In the initial portion of the process, the feed gas oxygen concentration is adjusted to maintain the desired value of the ethylene oxide production parameter, i.e., to maintain AEO(t) within the above-referenced range of values. This mode of operation is particularly useful when the desired reaction temperature is too low to be achieved because of reactor cooling circuit limitations.

In Table I, week 1 consists of 7 days (168 hours) and starts at t-t0=0. The first aging period consists of weeks 1-4. The second aging period starts at week 5. Prior to week 5, the reference feed gas composition values, the reference reaction condition values, and the efficiency-maximizing, optimum overall catalyst chloriding effectiveness value are reviewed. Since this review occurs every four weeks, the aging period counter is incremented every four weeks. For the catalyst of this example, the value of Z*opt is determined in accordance with Equation (8). In the first aging period, the initial feed gas oxygen concentration is increased from 6.0 to 6.36 vol. % to maintain the desired value of AEO (FIG. 3A-B). The second period consists of weeks 5-8. At week 5, Z* is increased from 3.07 to 3.14. As the catalyst continues to suffer aging-related activity losses, the feed gas oxygen concentration is progressively incremented in steps of about 0.1-0.2 vol. %. Catalyst deactivation is counteracted by increasing Cm up to week 16 (aging period 4). At week 17, any further increase to the feed gas oxygen concentration would exceed the maximum desired value of 7.5 vol. %. Therefore, starting with aging period 5, the catalyst aging is counteracted by increasing the reaction temperature. In both cases, Z* adjustments are made in order achieve the desired level of underchlorided overall catalyst chloriding effectiveness (Z*/Z*opt; FIG. 3E) and concomitant selectivity penalty (Asel; FIGS. 3F3G).

The data of Table I is calculated from the data presented in FIGS. 3A-3G. Referring to FIG. 3A, and based on Equations (6) through (9) from which the table data was generated, it can be seen that during each aging period (n) the value of the ethylene oxide production parameter (AEO) is maintained within a range of 2.24±0.02 vol. %. The average value of AEO is 2.243 vol. % (FIG. 3A). The value of the selectivity penalty (Asel) is maintained within a range of 0.16±0.04%, with an average value of 0.156% (FIG. 3F). The value of Z*/Z*opt is maintained within a range of 87.8±1.2%, with an average value of 87.8% (FIG. 3E). For the first 16 weeks, the reaction temperature remains constant (225° C.; FIG. 3C), and the feed gas oxygen concentration (FIG. 3B) is increased in steps. After week 16, with Coe at 7.50 vol. %, temperature is used to maintain the desired value of the ethylene oxide production parameter. Each time there is an increase in either the feed gas oxygen concentration, or the reaction temperature, the value of AEO undergoes a step increase (FIG. 3A), and Z*/Z*opt drops (FIG. 3E). Each time there is an increase in Z* (FIG. 3D), the value of AEO undergoes a relatively small step increase (FIG. 3A), and the selectivity penalty undergoes a relatively large decrease (FIG. 3F). The decrease in the selectivity penalty on increasing Z* (FIG. 3F) in combination with the absence of a parabolic minimum at Z*/Z*opt<89% in the plot of Asel vs. Z*/Z*opt (FIG. 3G) indicate that the operations are at a level of underchlorided overall catalyst chloriding effectiveness that is desired for reducing the aging-related deactivation of a high-selectivity, rhenium-promoted, silver ethylene oxide catalyst relative to that for operations for an extended period of time at Z*/Z*opt>100%.

TABLE I

| aging | | T | CO2 | | adjustment | E0_{produced} | E0 produced |
|---|---|---|---|---|---|---|---|
| period | week | (° C.) | (vol. %) | Z* | to operation | (t/m³/wk.) | (lct/m³1 4 wk.) |
| 1 | 1 | 225 | 6 | 3.07 | — | 44.37 | |
| 1 | 2 | 225 | 6.1 | 3.07 | CO2 | 44.37 | |
| 1 | 3 | 225 | 6.2 | 3.07 | CO2 | 44.36 | |
| 1 | 4 | 225 | 6.36 | 3.07 | CO2 | 44.56 | 0.178 |
| 2 | 5 | 225 | 6.36 | 3.14 | Z* | 44.29 | |
| 2 | 6 | 225 | 6.5 | 3.14 | CO2 | 44.42 | |
| 2 | 7 | 225 | 6.65 | 3.14 | CO2 | 44.56 | |
| 2 | 8 | 225 | 6.65 | 3.21 | Z* | 44.30 | 0.178 |
| 3 | 9 | 225 | 6.8 | 3.21 | CO2 | 44.44 | |
| 3 | 10 | 225 | 6.95 | 3.21 | CO2 | 44.57 | |
| 3 | 11 | 225 | 6.95 | 3.26 | Z* | 44.29 | |
| 3 | 12 | 225 | 7.15 | 3.26 | CO2 | 44.57 | 0.178 |
| 4 | 13 | 225 | 7.15 | 3.31 | Z* | 44.29 | |
| 4 | 14 | 225 | 7.35 | 3.31 | CO2 | 44.55 | |
| 4 | 15 | 225 | 7.35 | 3.36 | Z* | 44.28 | |
| 4 | 16 | 225 | 7.5 | 3.36 | CO2 | 44.38 | 0.178 |
| 5 | 17 | 225.2 | 7.5 | 3.4 | TZ* | 44.44 | |
| 5 | 18 | 225.4 | 7.5 | 3.4 | T | 44.45 | |
| 5 | 19 | 225.6 | 7.5 | 3.4 | T | 44.46 | |
| 5 | 20 | 225.8 | 7.5 | 3.4 | T | 44.47 | 0.178 |
| 6 | 21 | 226.05 | 7.5 | 3.4 | T | 44.57 | |
| 6 | 22 | 226.05 | 7.5 | 3.48 | Z* | 44.34 | |
| 6 | 23 | 226.3 | 7.5 | 3.48 | T | 44.44 | |
| 6 | 24 | 226.5 | 7.5 | 3.48 | T | 44.46 | 0.178 |
| 7 | 25 | 226.7 | 7.5 | 3.48 | T | 44.48 | |
| 7 | 26 | 226.9 | 7.5 | 3.48 | T | 44.51 | |
| 7 | 27 | 227.13 | 7.5 | 3.48 | T | 44.58 | |
| 7 | 28 | 227.13 | 7.5 | 3.56 | Z* | 44.37 | 0.178 |
| 8 | 29 | 227.35 | 7.5 | 3.56 | T | 44.43 | |
| 8 | 30 | 227.55 | 7.5 | 3.56 | T | 44.46 | |
| 8 | 31 | 227.75 | 7.5 | 3.56 | T | 44.49 | |
| 8 | 32 | 227.95 | 7.5 | 3.56 | T | 44.52 | 0.178 |

### Example 2

Aging data of a high selectivity catalyst at different ethyl chloride concentrations.

### Catalyst Synthesis

The catalyst carrier is a high purity alpha-alumina carrier obtained from Saint-Gobain NorPro in the shape of a penta-ring. The surface area is 1.16 m²/g, the pore volume is 0.70 cm³/g, and the packing density is 524 kg/m³. The alpha-alumina content of the carrier is greater than about 80 weight percent. The acid-leachable alkali metals (particularly lithium, sodium, and potassium) are less than about 30 parts per million by weight. In addition, the carrier contains zircon in an amount of 21 parts per thousand by weight. These weight compositions are calculated relative to the total weight of the carrier.

Eight solutions are prepared prior to the synthesis of the high-selectivity catalyst. The silver impregnation solution is prepared in accordance with the procedure described in US 2009/0177000 A1 and contains, by weight, 27% silver oxide, 18% oxalic acid dihydrate, 17% ethylenediamine, 6% monoethanolamine, and 31% water. Seven additional solutions are prepared by dissolving precursors into deionized water, one precursor for each solution. The seven precursors are manganese nitrate (Mn(NO3)2), diammonium ethylenediaminetetraacetic acid ((NH4)2H2(EDTA)), cesium hydroxide (CsOH), lithium acetate (LiOCOCH3), sodium acetate (NaOCOCH3), ammonium sulfate ((NH4)2SO4), and ammonium perrhenate (NH4Re04). The manganese and EDTA solutions are pre-mixed prior to addition into the silver solution. The EDTA/Mn mole fraction of this premix is 2.35 mol/mol. The ammonium perrhenate (NH4ReO4) promoter solution is prepared by dissolving the salt in deionized water that is gently heated to 4050° C. while stirring.

The catalyst is synthesized by vacuum impregnation. The carrier is used as received. The synthesis is carried out in two impregnations. The first impregnation is conducted using the unpromoted silver impregnation solution. The wet impregnated pills are then drained of excess solution and roasted in air at approximately 530° C. for 2.5 minutes. After the first impregnation and roasting, a second vacuum impregnation is carried out to add additional silver as well as catalyst promoters. The solution for the second impregnation is prepared by adding the individual promoter solutions to the silver solution in quantities that are pre-calculated to create the desired promoter composition on the finished catalysts. After the second impregnation, the pills are again drained and then roasted at 500° C. for 10 minutes in an air oven. The catalyst is cooled and weighed to estimate the loadings of silver and the impregnated promoters. The final catalyst contains 33.9 wt. % silver, and the promoter impregnation loadings are 779 ppm cesium, 45 ppm lithium, 54 ppm sodium, 103 ppm sulfate, 863 ppm rhenium, and 115 ppm manganese.

### Catalyst Testing

Six allotments are taken from the high selectivity catalyst, 500 mg/lot, and loaded into a set of 6 parallel microreactors. The catalyst testing is performed in the 6 parallel microreactors under simultaneous operation for 28 days at a reaction temperature of 250° C. and a reaction pressure of 1480 kPa (gauge pressure=1380 kPa) with a continuous flow of feed gas comprising ethylene (29.6 vol. %), ethane (1.95 vol. %), oxygen (7.4 vol. %), carbon dioxide (1.3 vol. %), and ethyl chloride (ranging from 10 to 24 ppmv). The chlorination of the catalyst is defined by a parameter Z* that is calculated as follows:

Z*=ECL(ppmv)(C2H6+0.01·C2H4) (10)

where, ECL is the feed gas concentration of ethyl chloride (ppmv), C2H6 is the feed gas concentration of ethane in mole percent, and C2H4 is the feed gas concentration of ethylene in mole percent. Prior to the aging tests, the catalysts are activated for six days at GHSV=17600/hr. All six reactors follow a Z* program consisting of four Z* plateaus. The initial Z* value is Z*=8.3. At 3.1 days, the Z* value is set to Z*=4.7. At 3.9 days, the Z* value is set to Z*=6.4, and at 4.9 days, the Z* value is set to Z*=10.6.

To evaluate the influence of the extent of chlorination on aging rates, six aging experiments are performed simultaneously in the set of six reactors. Of the six experiments, two are at Z*=6.0, two are at Z*=8.0, and two are at Z*=10.5. At 5.3 days, the Z* values are changed to these values. At 5.9 days, the feed gas flow rates are adjusted such that the outlet AEO is around 2 vol. % for each of the reactors. Then each of the six aging tests are carried out at constant GHSV, reaction temperature, reaction pressure, and feed gas composition (constant Z*). At 8.0 days, the reactors achieve a steady-state performance in terms of outlet AEO and selectivity.

### Results

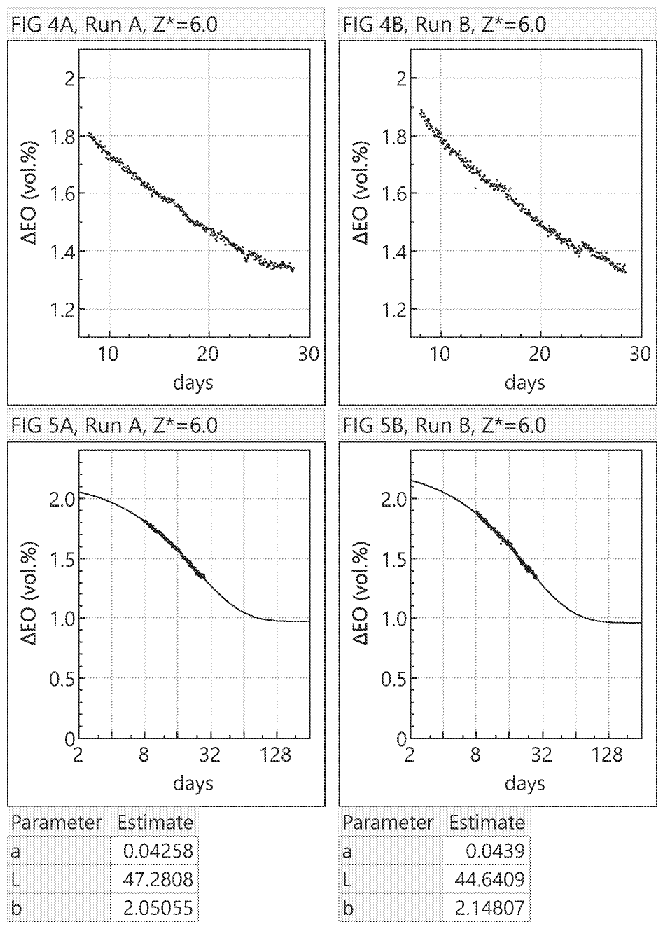

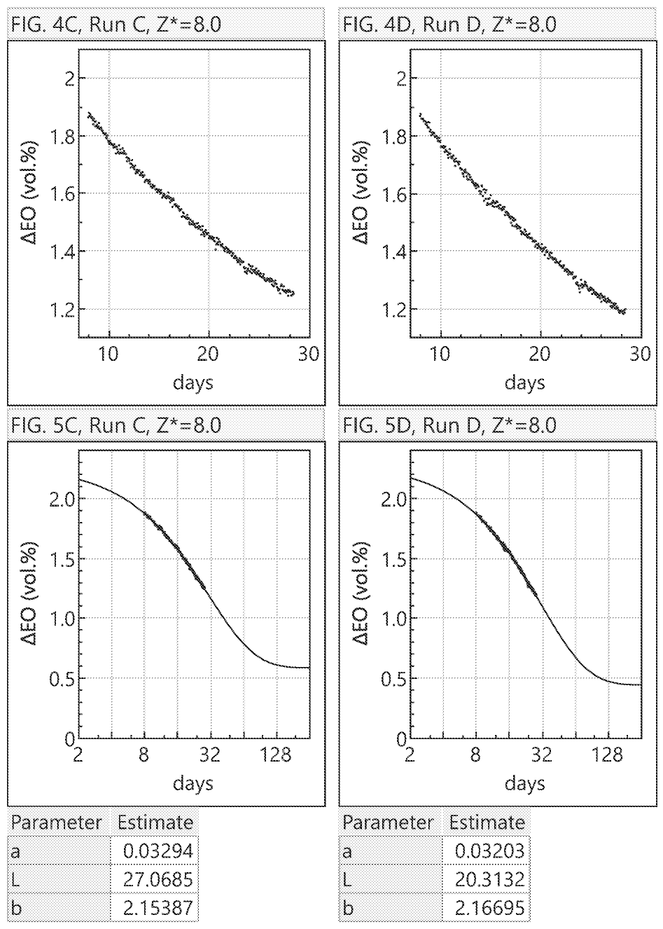

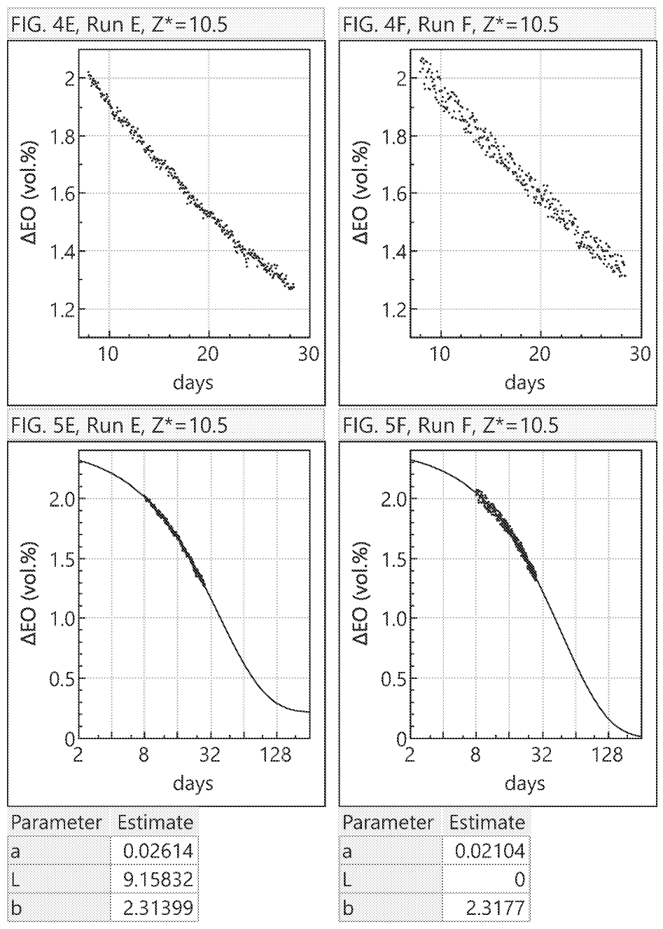

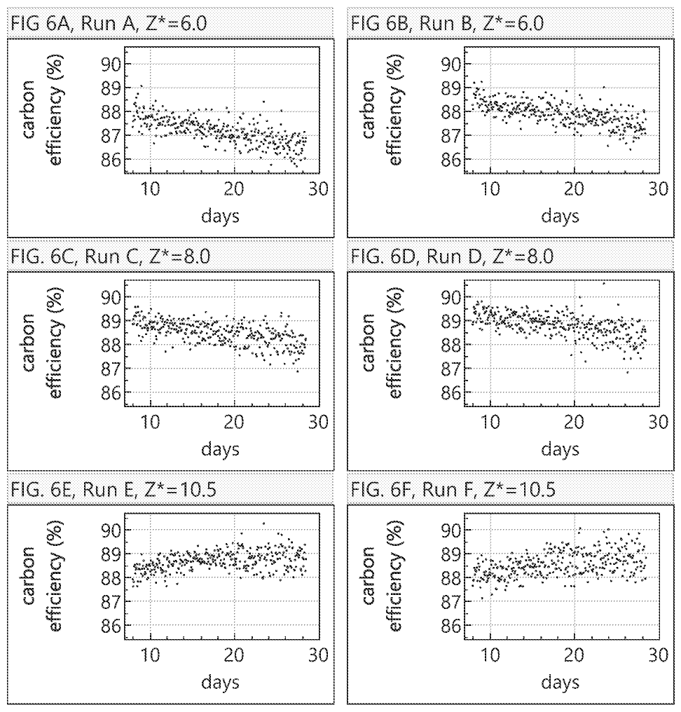

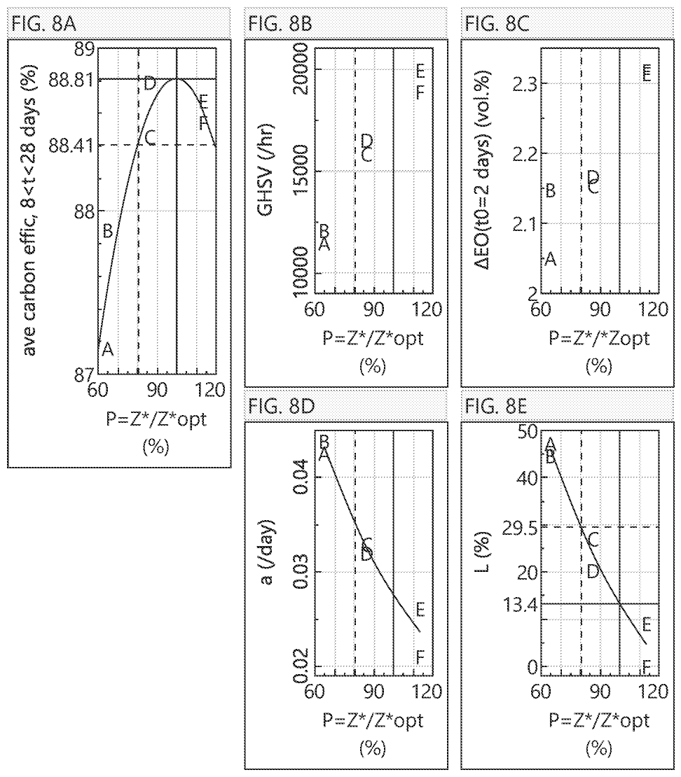

Results of the six aging tests are shown for a high efficiency catalyst. The catalyst activities are shown in FIGS. 4A-4F. Catalyst activity at time>28 days is determined by fitting first-order general power law equation (GPLE) models to the experimental results at 8<time<28 days, as shown in FIGS. 5A-5F, where time is plotted on base-two logarithmic axes. The catalyst efficiencies are given in FIGS. 6A-6F. As shown in FIG. 8A, a fit of the average catalyst efficiency against Z* indicates that the peak in carbon efficiency occurs at Z*=Z*opt=9.23. Hence, these experiments span a range of 65.0<Z*/Z*opt<113.8%.

For cases where the reaction parameters are held fixed (temperature, pressure, gas flow, and inlet composition), the GPLE models for the change in catalyst activity (y) with time (t) can be given as

dy=−a·(y−L·yo)°·dt, (11)

where 0 is the GPLE order parameter (0>1), a is the rate constant parameter (days'), yo is the activity at a chosen reference time to, and L·yo is the activity in the limit as t approaches infinity, where O<L<100%. Relative to the activity at time t=to, the loss of activity in the limit as t approaches infinity is 100%-L, where L is in percent, non-negative, and less than 100%.

Herein, the GPLE models use the equations listed below, with the reference time taken as to=2 days.

$$( for 0 = 1 ) y ( t ) = AEO ( t ) = AEO ( to ) [ ( 100 \% - L ) ( exp ( - a ( t - to ) ) + L ] \qquad ( 12 )$$

$$( for 0 > 1 ) y ( t ) = AEO ( t ) = {[ ( 0 - 1 ) [ a ( t - to ) \pm ( AEO ( to ) { ( 1 - L )}^{1 - o} ] / ( 0 - 1 ) ]}^{( 1 - 41 - 0 )} + AEO ( to ) L \qquad ( 13 )$$

For each experiment, once 0 is chosen, three model parameters (a, AEO(to), and L) are determined by nonlinear least-squares fit to the experimental data.

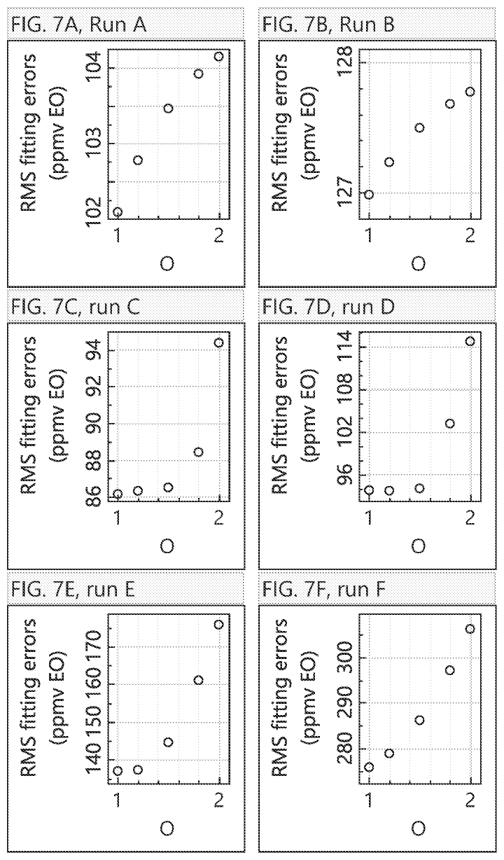

The data of FIGS. 4A-4F are fit to GPLE models with order parameters (0) of 1.0, 1.2, 1.5, 1.8, and 2.0. The quality of fits is particularly good for the first order GPLE model (0=1), as shown in FIGS. 5A-5F, but becomes progressively inferior with increasing values of 0, as shown in FIGS. 7A-7F, where the ordinates show root mean square errors of the fits as a function of 0.

FIGS. 8A-8E show the dependence of catalyst metrics on the level of gas phase promotion relative to the fixed temperature optimum level of gas phase promotion, i.e., P=Z*/Z*opt. Vertical lines are drawn at P=80.5% (dashed) and P=100% (solid). FIG. 8A shows the average catalyst efficiency as a function of P for 8<t<28 days. The peak efficiency occurs at Z*=9.23. The P values for the experiments are 65.0% (example A and B), 86.7% (examples C and D), and 113.8% (examples E and F). At P=80.5%, the penalty in catalyst selectivity is only 0.4% (88.41 vs. 88.81% at P=100%).

In order to compensate for the increase in activity with increasing Z*, GHSV was increased with increasing Z*. Then, for each of the six examples, GHSV was held fixed over the duration of the activity aging segment of the experiments (5.9<t<28 days). FIG. 8B shows the gas hourly space velocities (GHSV).

FIG. 8E shows the catalyst activity asymptotic limit (L) parameters as a function of P for the GPLE (0=1) model. Operating at the fixed temperature optimum Z* value (P=100%, Z=Z*opt) gives an L value of 13.4%. Operating at Z* values less than Z*opt yields values of L that are greater than 13.4%. In the case of P=80.5%, L is more than twice the value of L at P=100%. The other two GPLE (0=1) parameters are shown as a function of time in FIG. 8C [AEO(to)] and FIG. 8D [the rate constant parameter, a].

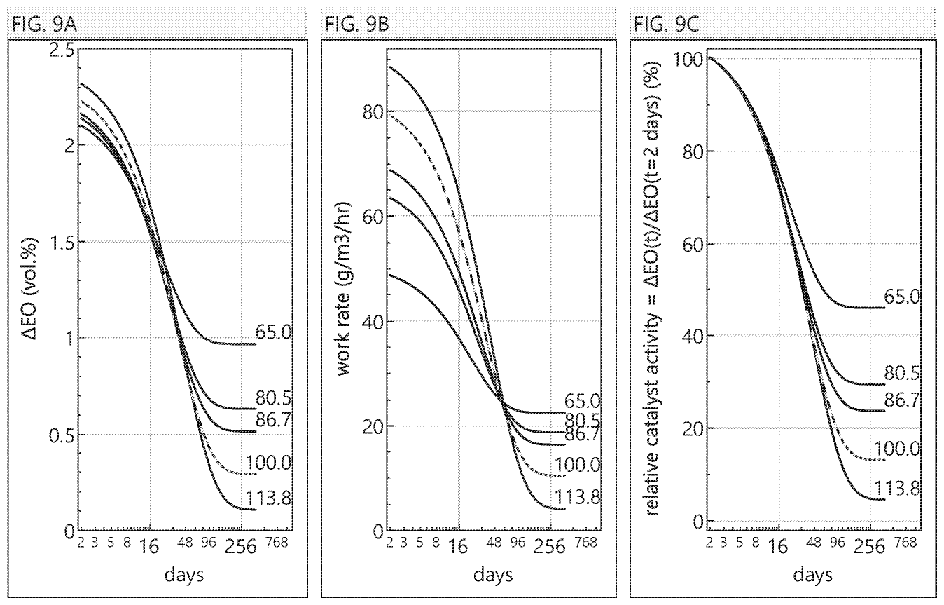

FIGS. 9A-9C show the trends over time for AEO, work rate and the relative catalyst activity, given as AEO/AEO(t=2 days). The trends are shown for five values of P=Z*/Z*opt, ranging from 65 to 114%. These trends are plotted against time, where time is on a logarithmic axis. The trends are generated using the GPLE(O=1) model and using parabolic fits of the parameters as a function of P=Z*/Z*opt for GHSV (FIG. 8B), AEO(to) (FIG. 8C), a (FIG. 8D), and L (FIG. 8E). As a result, the trends are generated for operations at a constant reaction temperature of 250° C. and at a constant reaction pressure of 1480 kPa. As shown in FIGS. 9A-9B, after 256 days, the aging-related decreases in both AEO and the work rate are minimal. As shown in FIG. 9C, for the four cases with 80<P<114%, for t<28 days, the relative catalyst activity is insensitive to the level of gas phase promotion. After 100 days, the values of AEO, work rate, and relative catalyst activity for the three cases at P<90% are larger than the respective values for the two cases at P>100%. This indicates that sub-optimal chloriding beneficially decreases activity aging effects relative to both fixed temperature optimum chloriding and supra-optimal chloriding. For each of the three cases with P<90%, in comparison to the two P>100% cases, the, operation at the sub-optimal Z* value shows a decrease in aging-related AEO and work rate losses at constant reaction temperature over a year long period.

The foregoing demonstrates an unexpected activity aging advantage and longer catalyst life for operation at P<100% while incurring only a small penalty in initial catalyst activity and selectivity. Without wishing to be bound by any particular theory, it is believed that this advantage in useful catalyst life is due to a combination of (a) avoiding excessive surface chloride, and (b) reducing the rate of sintering. Operating at chloriding levels of P<85% may allow for essentially perpetual operation at high selectivity given appropriate selection of operational parameters; e.g., low temperature and reduced work rate.

1. A method for reducing aging-related deactivation of a high-efficiency, rhenium-promoted silver catalyst in a process for manufacturing ethylene oxide, wherein at the start of a first catalyst aging period the process has a first efficiency-maximizing, optimum overall catalyst chloriding effectiveness value at:
a) a first reference feed gas composition, comprising ethylene at a first reference feed gas concentration value of ethylene, oxygen at a first reference feed gas concentration value of oxygen, water at a first reference feed gas concentration value of water, and at least one organic chloride at a first reference feed gas concentration value of the at least one organic chloride; and
b) a first set of reference reaction condition values, comprising a first reference reaction temperature value, a first reference gas hourly space velocity value, and a first reference reaction pressure value,
the method comprising:
reacting a first feed gas composition over the catalyst during the first catalyst aging period at:
i) a first overall catalyst chloriding effectiveness that never exceeds 95 percent of the first efficiency-maximizing, optimum overall catalyst chloriding effectiveness value during the first catalyst aging period; and
ii) a first set of reaction conditions, comprising a first reaction temperature that is no less than the first reference reaction temperature value and which varies from the first reference reaction temperature value by no more than +3° C. during the first catalyst aging period, the first reference reaction pressure value, and the first reference gas hourly space velocity value,
wherein, the first feed gas composition comprises:
aa) oxygen at a first feed gas concentration of oxygen that is no less than the first reference feed gas concentration value of oxygen, and which varies from the first reference feed gas concentration value of oxygen by no more than +1.2 volume percent during the first catalyst aging period,
bb) ethylene at a first feed gas concentration of ethylene, and
cc) water at a first feed gas concentration of water that is no greater than the first reference feed gas concentration value of water, and which varies from the first reference feed gas concentration value of water by no more than −0.4 volume percent during the first catalyst aging period,
wherein the first catalyst aging period is no less than 0.03 kt ethylene oxide/m³ catalyst.

2. The method of claim 1, wherein at the start of a subsequent catalyst aging period the process has a subsequent efficiency-maximizing, optimum overall catalyst chloriding effectiveness value at:
a. a subsequent reference feed gas composition, comprising ethylene at a subsequent reference feed gas concentration value of ethylene, oxygen at a subsequent reference feed gas concentration value of oxygen, water at a subsequent reference feed gas concentration value of water, and at least one organic chloride at a subsequent reference feed gas concentration value of the at least one organic chloride; and
b. a subsequent set of reference reaction condition values, comprising a subsequent reference reaction temperature value, a subsequent reference gas hourly space velocity value, and a subsequent reference reaction pressure value,
the method further comprising:
reacting a subsequent feed gas composition over the catalyst during the subsequent catalyst aging period at:
(i) a subsequent overall catalyst chloriding effectiveness that never exceeds 95 percent of the subsequent efficiency-maximizing, optimum overall catalyst chloriding effectiveness value during the subsequent catalyst aging period; and
(ii) a subsequent set of reaction conditions, comprising a subsequent reaction temperature that is no less than the subsequent reaction temperature reference value and which varies from the subsequent reference reaction temperature value by no more than +3° C. during the subsequent catalyst aging period, the subsequent reference reaction pressure value, and the subsequent reference gas hourly space velocity value,
wherein, the subsequent feed gas composition comprises:
(aa) oxygen at a subsequent feed gas concentration of oxygen that is no less than the subsequent reference feed gas concentration value of oxygen, and which varies from the subsequent reference feed gas concentration value of oxygen by no more than +1.2 volume percent during the subsequent catalyst aging period,
(bb) ethylene at a subsequent feed gas concentration of ethylene, and
(cc) water at a subsequent feed gas concentration of water that is no greater than the subsequent reference feed gas concentration value of water, and which varies from the subsequent reference feed gas concentration value of water by no more than −0.4 volume percent during the subsequent catalyst aging period,
wherein the subsequent catalyst aging period is no less than 0.03 kt ethylene oxide/m³ catalyst.

3. The method of claim 2, wherein the subsequent set of reaction conditions and the
subsequent feed gas composition correspond to a desired value of an ethylene oxide production parameter.

4. The method of claim 1, wherein the first catalyst aging period is no less than 0.06 kt ethylene oxide/m³ of the catalyst.

5. A method according to claim 1, wherein the overall catalyst chloriding effectiveness value is represented by the formula:
Z*=(ECL+2·EDC+VCL)(C2H6+0.01·C2H4)
wherein, ECL is the concentration of ethyl chloride in the feed gas in ppmv;
EDC is the concentration of ethylene dichloride in the feed gas in ppmv;
VCL is the concentration of vinyl chloride in the feed gas in ppmv; C2H6 is the concentration of ethane in the feed gas in mole percent; and C2H4 is the concentration of ethylene in the feed gas in mole percent.

6. The method of claim 5, wherein the first efficiency-maximizing, optimum overall catalyst chloriding effectiveness value is represented as Z*opt(1) and is a value of Z* at which the efficiency is at a maximum at the first reference feed gas composition and the first set of reference reaction condition values.

7. The method of claim 6, wherein during the first catalyst aging period, the first overall catalyst chloriding effectiveness never exceeds a value of 95 percent of Z*opt(1).

8. A method according to claim 1, wherein during the first catalyst aging period, the first overall catalyst chloriding effectiveness never falls below 75 percent of Z*opt(1).

9. A method according to claim 1, wherein the first efficiency-maximizing, optimum overall catalyst chloriding effectiveness value corresponds to a first maximum efficiency, and during the first catalyst aging period, the process has a first efficiency that never falls below the first maximum efficiency by more than 0.5%.

10. A method according to claim 1, further comprising adjusting one selected from the first reaction temperature and the first feed gas concentration of oxygen during the first catalyst aging period to maintain a desired value of an ethylene oxide production parameter, or to achieve a new value of an ethylene oxide production parameter.

11. The method of claim 10, wherein the step of adjusting one selected from the first reaction temperature and the first feed gas concentration of oxygen during the first catalyst aging period comprises adjusting the first reaction temperature.

12. The method of claim 10, wherein the step of adjusting one selected from the first reaction temperature and the first feed gas concentration of oxygen during the first catalyst aging period comprises adjusting the first feed gas concentration of oxygen.

13. A method according to claim 1, wherein the ethylene oxide production parameter is one selected from the group consisting of ethylene oxide yield, ethylene oxide reactor product concentration, ethylene conversion, oxygen conversion, ethylene oxide work rate, and ethylene oxide production rate.

14. A method according to claim 1, further comprising the step of selecting one or more values of the first overall catalyst chloriding effectiveness.

15. A method in accordance with claim 1, wherein the first reaction temperature ranges from about 200° C. to about 300° C.

16. A method in accordance with claim 1, wherein the first reaction pressure value ranges from about 500 kPa to about 3.0 MPa.

17. A method in accordance with claim 1, wherein the first gas hourly space velocity value is at least about 3000 hr⁻¹.

18. A method in accordance with claim 1, wherein during the first catalyst aging period, the first overall catalyst chloriding effectiveness never falls below a Z* lower limit value of about 1 and never exceeds a Z* upper limit value of about 20.
